(19) United States
(12) Patent Application Publication
(10) Pub. No.: US 2006/0292083 A1
(43) Pub. Date: Dec. 28, 2006

(54) Inhalation compositions with high drug ratios

(76) Inventor: Xian-Ming Zeng, Mitcham (GB)

Correspondence Address: IVAX CORPORATION, 4400 Biscayne Boulevard, Miami, FL, 33137, US

(21) Appl. No.: 10/646,362

(22) Filed: Aug. 21, 2003

(30) Foreign Application Priority Data
Aug. 21, 2002 (GB) 0219512.1

(51) Int. Cl. A61K 9/14; A61L 9/04

(52) U.S. Cl. 424/46

(57) ABSTRACT

The invention provides a dry powder inhalation composition comprising, at least 0.25% by weight of the composition of an active ingredient with a particle size of less than 10 microns in diameter and a pharmaceutically acceptable particulate carrier with a particle size of less than 250 microns in diameter. Also disclosed are methods for use of the compositions of the invention with dry powder inhalers for therapeutic treatments.

### CROSS-REFERENCE TO RELATED APPLICATIONS

This application claims priority to United Kingdom Patent Application No.: 0219512.1 filed on Aug. 21, 2002.

### TECHNICAL FIELD OF THE INVENTION

This invention relates to dry powder inhalation compositions, their preparation and use. In particular, it is concerned with formulations of the medicament formoterol and pharmaceutically acceptable derivatives thereof mixed with particulate lactose.

### BACKGROUND OF THE INVENTION

In order to be able to be inspired into the key target sites in the lungs of patients, inhalation drugs are typically provided in microns in diameterized form with average particle sizes of up to 10 microns in diameter. A number of devices have been developed for assisting the delivery of such medicaments into the lungs of patients. In one sort of device, a dry powdered inhaler (DPI) device, the medicament to be inhaled is dispensed into an air stream produced by the inspiratory action of the patient. A large number of such devices have been developed. The device may be a single dose device (e.g., where drug is dispensed from a pre-metered dosage means, such as a capsule) or multidose (where the drug is stored in a reservoir and then metered prior to dispersal in the air stream, or where the drug is pre-metered and stored in multiple dosage packs, such as blisters). In a number of DPI devices, the particulate drug is mixed with an excipient powder of larger average particle size and the drug particles are blended with the excipient to create a generally homogenous mixture. The larger particle size of the excipient results in the powder mixture being flowable, and the homogeneity of the mixture enable it to be metered into accurately measurable doses. This is of particular importance when only very small quantities of the drug are required in a dose. Excipient powders of this kind, pharmaceutical powder compositions for inhalation utilizing such excipients are described, for example, in U.S. Pat. No. 3,957,965.

The accurate metering of highly potent inhalant drugs causes particular problems, as the quantity of medicament in the composition relative to that of the carrier is likely to be particularly small (less than I part of drug to 50 parts of carrier). This is exemplified by the medicament formoterol, which is often administered to patients at a dose of less than 60 micrograms (doses may be as small as 6 micrograms).

U.S. Pat. No. 6,199,607 to Trofast describes a multi-step process for preparing a dry powder formoterol composition. The process as described includes the mixing of the components followed by micronization of the blend. The micronized particles were subsequently treated to remove amorphous areas in their crystal structure. The particles are then agglomerated, sieved, and spheronized, followed by a second sieving, spheronization and sieving.

What are needed then are simple methods for producing dry powder medicaments of high drug ratio, which maintain desirable flow and deposition characteristics following dispersion.

### SUMMARY OF THE INVENTION

The invention provides a dry powder inhalation composition comprising, at least 0.25% (by weight of the composition) of an active ingredient with a particle size of less than 10 microns in diameter and a pharmaceutically acceptable particulate carrier with a particle size of less than 250 microns in diameter. Also disclosed are methods for use of the compositions of the invention with dry powder inhalers.

Hence, dry powder inhalation compositions of a particulate medicament (e.g., formoterol) and lactose of defined particulate size and proportions are described which are easier to handle, and can be readily filled into the reservoir of a multidose dry powder inhaler (MDPI), (see, for example, WO 92/10229). Additionally, these compositions are more accurately metered and provide more uniform and consistent dispersions when dispensed by MDPI devices. Certain compositions may also be more stable

Another aspect of the invention provides for a multidose dry powder inhaler comprising the inhaler and a composition according to the invention.

In yet another aspect of the invention, methods for the administration of a particulate medicament, comprising inhalation of a composition of the invention from a multidose dry powder inhaler, are provided.

The invention additionally invention provides a method for the administration of a therapeutically effective amount of compositions of the invention, for the treatment of conditions responsive to the active ingredient of choice.

### BRIEF DESCRIPTION OF THE DRAWINGS

FIG. 1: Graphical representation of fine particle fraction for a formoterol formulation (n=32, error bars denote standard deviation).

### DETAILED DESCRIPTION OF THE INVENTION

The invention provides a dry powder inhalation composition comprising, at least 0.25% by (weight of the composition) of an active ingredient with a particle size of less than 10 microns in diameter and a pharmaceutically acceptable particulate carrier with a particle size of less than 250 microns in diameter. Also disclosed are methods for use of the compositions of the invention with dry powder inhalers.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001).

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used in this specification, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%.

As used herein, unless specifically indicated otherwise, the word “or” is used in the “inclusive” sense of “and/or” and not the “exclusive” sense of “either/or.”

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

An aspect of the invention provides a dry powder inhalation composition comprising, at least 0.25% (by weight of the composition) of an active ingredient with a particle size of less than 10 microns in diameter and a pharmaceutically acceptable particulate carrier with a particle size of less than 250 microns in diameter.

In some embodiments, the compositions comprise less than 10% (by of the composition) of an active ingredient. In other embodiments, the compositions comprise from about 0.26 to about 1% (by weight of the composition) of an active ingredient, while in yet other embodiments the compositions comprise from about 0.265 to about 0.5% (by weight of the composition) of an active ingredient.

In some embodiments, the pharmaceutically acceptable particulate carriers are disaccharides or polysaccharides. In other embodiments, the particulate carrier is lactose, while in yet other embodiments the particulate lactose is alpha lactose monohydrate. In general, the particle size of the lactose should be such that it can be entrained in an air stream but not deposited in the key target sites of the lung. Accordingly, in some embodiments, lactose with a mean particle size of less than 40 microns in diameter is excluded. In other embodiments, the particulate carrier has a VMD of from about 50 to about 250 microns in diameter. The VMD of the carrier is from about 50 to about 60 μm in some embodiments, from about 60 to about 90 microns in diameter or from about 90 to about 150 microns in diameter in yet other embodiments. Particle size may be determined using laser light scattering (Sympatec GmbH, Claasthal-Zellerfeld, Germany).

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

The compositions according to the invention are optionally formulated in a pharmaceutically acceptable vehicle with any of the well-known pharmaceutically acceptable medically inert moiety such as carriers, including diluents, excipients, surfactants, and flavourings (see Remington's Pharmaceutical Sciences, 18^{th} Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. See also Zeng, et al., Particulate Interations in Dry Powder Formulations of Inhalation, Taylor & Francis, London, 2001.

As used herein, “medicament” or “active ingredient” (used interchangeably) is meant to encompass active pharmaceuticals appropriate for inhalation therapy in dry powder form. Representative, non-limiting examples include bronchodilators (e.g., epinephrine, metaproterenol, terbutaline, albuterol, and the like), anticholinergic agents (e.g., ipratropium bromide), xanthines (e.g., dyphylline, aminophylline), inhalant corticosteroids (e.g., flunisolide, beclomethasone, budesonide, and the like), or β-2 adrenergic receptor agonists (e.g., salmeterol and formoterol).

In some embodiments, the active ingredient is formoterol or a pharmaceutically acceptable derivative thereof.

For example, where the medicament is formoterol, the active ingredient may be in any isomeric form or mixture of isomeric forms, for example a pure enantiomer, particularly the R,R-enantiomer, a mixture of enantiomers, a racemate or a mixture thereof. Pharmaceutically acceptable derivatives of formoterol include pharmaceutically acceptable salts, in particular acid addition salts with inorganic acids such as hydrochloric acid, hydrobromic acid, sulphuric or phosphoric acid. The salt may also be with an organic acid such as acetic, succinic, maleic, fumaric, citric, tartaric, lactic or benzoic. The active ingredient and pharmaceutically acceptable derivatives thereof may exist in the form of a solvate, in particular a hydrate. A preferred form of active ingredient for use in the invention is formoterol fumarate, especially formoterol fumarate di-hydrate, conveniently in its racemic form. Formoterol, salts and hydrates thereof and salt hydrates thereof as described above may be prepared by known methods, for example as described in U.S. Pat. No. 3,994,974 or U.S. Pat. No. 5,684,199.

In some embodiments, the active ingredient is present in the dry powder composition at an amount that is less than 10% by weight of the composition, in other embodiments less than 2% by weight of the composition, and in yet other embodiments, the active ingredient is less than 1% by weight of the composition. Compositions according to the invention may contain from about 0.26% to about 1% (by weight of the composition) of the active ingredient. In some instances, the amount of active ingredient ranges from about 0.265 to about 0.5% by weight of the composition. The actual amount of active ingredient in the composition will depend to a large extent on the nature of the dry powder inhaler and the quantity of composition that is metered for each individual dose. Where a large dose of composition is metered, the proportion of the active ingredient in the dose will be reduced. Particularly dilute compositions are disclosed in WO 01/39745, for example, 0.02% by weight.

In some embodiments, the mean particle diameter of the active ingredient is up to 10 microns in diameter, while in other embodiments, the mean particle size is up to 5 microns in diameter. In yet other embodiments, the mean particle size ranges from about 1 to 5 about microns in diameter. The particle size of the active ingredient can be reduced to the desired level by conventional means, for example by grinding in a mill, for example, an air jet, ball or vibrator mill, by sieving, by crystallization, by spray-drying or by lyophilization.

As used herein, “up to”, when used in conjunction with a percentage particulates of a named size, is meant to require the presence of an amount other than zero of particles of the named size and that the named numeric percentage is the upper limit for the presence of particles of the named size.

The formulations of the compositions of the invention may conveniently be presented in unit dosage form and may be prepared by conventional pharmaceutical techniques. Such techniques include the step of bringing into association the compound of the invention and the pharmaceutically acceptable carrier(s), or an excipient. In general, the formulations are prepared by uniformly and intimately bringing into association the active ingredient with finely divided solid carriers, and then, if necessary, preparing discrete dosage units of the product.

The dry powder composition may be metered and filled into capsules, e.g., gelatin or hydroxypropyl methylcellulose capsules, such that the capsule contains a unit dose of active ingredient.

Doses of active ingredient to be held in accordance with the invention may be in general from 1 to 60 micrograms. When the active ingredient is formoterol fumarate dihydrate, the dose may be, for example, from 6 to 54 micrograms. Preferred doses are from 6 to 24 micrograms, especially the unit doses of 6 micrograms, 12 micrograms and 24 micrograms. These doses may be administered once or twice daily.

When the dry powder is in a capsule containing a unit dose of active ingredient, the total amount of composition will depend on the size of the capsules and the characteristics of the inhalation device with which the capsules are being used. However, characteristic total fill weights of dry powder per capsule are between 1 and 5 mg. In some embodiments, the dry powder inhalation composition is in a capsule containing from I to 25 mg of the composition.

Alternatively, the dry powder composition according to the invention may be filled into the reservoir of any multidose dry powder inhaler (MDPI), for example of the kind illustrated in WO 92/10229 (hereinafter referred to as the IVAX™ MDPI).

Compositions according to the invention may be readily prepared by blending the required amount of active ingredient with the required amount of particulate carrier of the desired particle size distribution.

Another aspect of the invention provides for a MDPI comprising the dry powder inhalation composition of the invention.

Another aspect of the invention provides a method for the administration of a particulate medicament, comprising inhalation of a composition of the invention from a multidose dry powder inhaler.

In yet another aspect, the invention provides a method for the administration of a therapeutically effective amount of compositions of the invention, for the treatment of conditions responsive to the medicaments of choice. Non-limiting examples of conditions include chronic obstructive pulmonary disease, asthma, late phase allergic responses, or pulmonary inflammations. In one embodiment, the condition being treated is chronic obstructive pulmonary disease.

The term “therapeutically effective amount” is used to denote treatments at dosages effective to achieve the therapeutic result sought. Furthermore, one of skill will appreciate that the therapeutically effective amount of the compositions of the invention may be lowered or increased by fine tuning and/or by administering more than one composition of the invention, or by administering a composition of the invention with another compound or composition. The invention therefore provides a method to tailor the administration/treatment to the particular exigencies specific to a given mammal.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLES

### Example 1

0.265 grams of formoterol (as the fumarate dihydrate salt) was blended with 99.735 grams of lactose with VMD or MMD of 89-110 microns in diameter and a geometric standard deviation (GSD) of 2.2-4.9. Blending was conducted using a tumbling mixing process (TURBULA™, Glen Creston, N.J., USA). The formoterol lactose blend was filled into the reservoir of an IVAX MDPI device.

The inhalers that contained the formulation were then tested for pharmaceutical performance under conditions specified in European Pharmacopoeia (2001) including uniformity of delivered dose and fine particle dose. Through-life dose delivery was measured using a dose unit sampling unit in conjunction with a critical flow controller model TPK, high capacity pump and flowmeter (Copley Scientific, Nottingham, U.K.) while fine particle dose (FPD) and fine particle fraction (FPF) were measured using a 5-stage liquid impinger MSL also from Copley Scientific.

The compositions gave excellent dose uniformity when used in association with an IVAX™ MDPI device, which produced all mean doses within 80-120% label claim and overall relative standard deviation (RSD) <15% (Table 1). The same products also result in over 40% drug particles having aerodynamic particle size <5 microns in diameter, suggesting that they are highly efficient in generating deeply inspirable drug. Typical in vitro deposition profiles are shown in Table 2.

TABLE 1
Dose Consistency Over Life of Ivax Formoterol
MDPI, Expressed as % Label Claim (LC)

| | Overall mean | % mean doses | % individual | % Individual |
|---|---|---|---|---|
| | in mcg | within | doses within | doses within |
| Strength | (RSD) | 85-115% LC | 80-120% LC | 75-125% LC |
| 6 mcg | 5.7 (13%) | 95 | 93 | 96 |
| (n = 930) | | | | |
| 12 mcg | 12.2 (10%) | 100 | 97 | 99 |
| (n = 500) | | | | |

(n = Number of doses. Ten doses from the beginning, middle and end of device life were collected from each inhaler).

TABLE 2
In Vitro Deposition Profiles of Formoterol From the Ivax MDPI

| Strength | RD (μg) | FPD (μg) | FPF (% RD) |
|---|---|---|---|
| 6 mcg | 5.0-5.9 | 2.4-2.8 | 48—48 |
| 12 mcg | 11.1-13.4 | 5.4-7.2 | 49-54 |

RD—Recovered dose
FPD—Fine particle dose
FPF—Fine particle fraction

### Example 2

10.6 grams of formoterol (as the fumarate dihydrate salt) was blended with 3989.4 grams of lactose with VMD or MMD of 70-120 microns in diameter and filled into the reservoir of a dry powder inhaler of the type illustrated in WO 92/10229. Four batches of blend were made and each was filled in the devices with a small and large dose cup sizes to give 6 mcg and 12 mcg strength products, respectively. Blending was conducted using a tumbling mixing process (TURBULA™, Glen Creston, N.J., USA). The formoterol lactose blend was filled into the reservoir of an IVAX™ MDPI device.

The inhalers that contained the formulation were then tested for pharmaceutical performance under conditions specified in European Pharmacopoeia (2001) including uniformity of delivered dose and fine particle dose. Through-life dose delivery was measured using a dose unit sampling unit in conjunction with a critical flow controller model TPK, high capacity pump and flowmeter (Copley Scientific, Nottingham, U.K.) while fine particle dose (FPD) and fine particle fraction (FPF) were measured using a 5-stage liquid impinger MSL also from Copley Scientific.

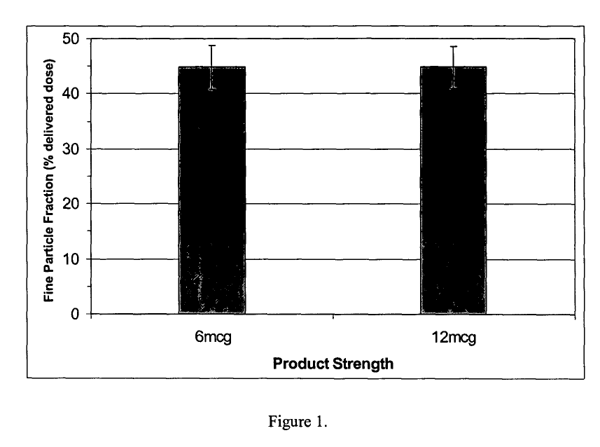

All four blends produced drug recovery within 95-105% target with relative standard deviation <5%, suggesting that the blending and handling process is efficient and reproducible (Table 3). After aerosolisation at the standard flow rate, the compositions gave excellent dose uniformity when used in association with the device of WO 92/10229, which produced all mean doses within 80-120% label claim (Table 3). The same products also result in over 40% drug particles having aerodynamic particle size <5 microns in diameter (FIG. 1), suggesting that they are highly efficient in generating deeply inspirable drug. There is no difference in the fine particle fraction of formoterol between the 6mcg and 12mcg strength products indicating a consistent performance of these products.

TABLE 3
Mean blend strength and delivered doses (mcg) of four batches
of blends containing 0.26-0.27% w/w formoterol (as the fumarate
dihydrate salt) in lactose monohydrate (Mean ± SD)

| | | Delivered Dose | Delivered Dose |
|---|---|---|---|
| Blend Batch | Formoterol Conc. | (6 mcg strength, | (12 mcg strength, |
| Number | (w/w %, n = 10) | n = 30) | n = 30) |
| EML-169 | 0.261 ± 0.011 | 5.7 ± 0.5 | 11.6 ± 1.1 |
| EML-170 | 0.274 ± 0.010 | 5.7 ± 0.6 | 11.7 ± 1.1 |
| EML-194 | 0.261 ± 0.011 | 5.9 ± 0.7 | 11.1 ± 1.2 |
| EML-197 | 0.260 ± 0.005 | 5.8 ± 0.8 | 11.3 ± 1.3 |

### Example 3

A blend of microns in diameterized medicament chosen from a group consisting of, but not limited to, bronchodilators (e.g., epinephrine, metaproterenol, terbutaline, albuterol, and the like), anticholinergic agents (e.g., ipratropium bromide), xanthines (e.g., dyphylline, aminophylline), inhalant corticosteroids (e.g., flunisolide, beclomethasone, budesonide, and the like), or β-2 adrenergic receptor agonists (e.g., salmeterol) is blended with lactose according to the methods described in Example 1. The resulting blend is introduced into an IVAX™ MDPI and then tested for pharmaceutical performance under the conditions specified in European Pharmacopoeia. The drug per actuation (DPA) is measured using a dose unit sampling unit while fine particle dose (FPD) and fine particle fraction (FPF) are measured using a 5-stage liquid impinger as previously described.

### Equivalents

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

1. A dry powder inhalation composition comprising,
(a) at least 0.25% (by weight of the composition) of an active ingredient with a particle size of less than 10 microns in diameter, and
(b) a pharmaceutically acceptable particulate carrier with a particle size of less than 250 microns in diameter.

2. The dry powder inhalation composition according to claim 1, wherein the composition comprises less than 10% (by weight of the composition) of the active ingredient.

3. The dry powder inhalation composition according to claim 1 or claim 2, wherein the composition comprises from about 0.26 to about 1% (by weight of the composition) of the active ingredient.

4. The dry powder inhalation composition according to claim 1, which comprises from about 0.265 to about 0.5% (by weight of the composition) of the active ingredient.

5. The dry powder inhalation composition according to claims 1 or 4, wherein the carrier is lactose.

6. The dry powder inhalation composition according to claims 1 or 4, wherein the active ingredient is formoterol or a pharmaceutically acceptable derivative thereof.

7. The dry powder inhalation composition according to claims 1 or 4, wherein the active ingredient is formoterol or pharmaceutically acceptable derivative thereof.

8. A capsule containing from 1 to 25 mg of a dry powder inhalation composition according to claims 1 or 4.

9. A MDPI comprising a reservoir containing the dry powder inhalation composition of claim 1 or 4.

10. A method for the treatment of chronic obstructive pulmonary disease by the step of administering the dry powder inhalation composition of claim 1 or 4.
